(19) United States
(12) Patent Application Publication
(10) Pub. No.: US 2003/0049657 A1
(43) Pub. Date: Mar. 13, 2003

(54) Use of primers containing non-replicatable residues for improved cycle-sequencing of nucleic acids

(76) Inventor: Joshua L. Cherry, Cambridge, MA (US)

Correspondence Address: Joshua L. Cherry, 2102 Biological Laboratories, 16 Divinity Ave., Cambridge, MA, 02138, US

(21) Appl. No.: 10/147,185

(22) Filed: May 15, 2002

Related U.S. Application Data
(62) Division of application No. 09/438,667, filed on Nov. 12, 1999, now abandoned.
(60) Provisional application No. 60/108,345, filed on Nov. 13, 1998.

(51) Int. Cl. C12Q 1/68; C12P 19/34

(52) U.S. Cl. 435/6; 435/91.2

(57) ABSTRACT

The present invention provides primers for use in cycle sequencing which are not subject to exponential amplification of undesired artifacts. Such primers cannot be replicated by the nucleic acid polymerases used in these reactions and, therefore, do not produce artifacts. Methods of linear amplification of a nucleic acid template using such primers are also provided.

### 1. RELATED APPLICATIONS

This application is a division of Ser. No. 09/438,667, filed Nov. 12, 1999. This application is related to and claims the benefit of U.S. Provisional Application Serial No. 60/108,345 of Joshua L. Cherry filed Nov. 13, 1998 and entitled “Use of Primers Containing Non-Replicatable Residues for Improved Cycle-Sequencing of Nucleic Acids,” which is incorporated herein by this reference.

### 2. FIELD OF THE INVENTION

The invention relates to primers for use in sequencing DNA. More particularly, the invention relates to primers that may be used for linear amplification of DNA but limit undesirable exponential amplification of artifacts and methods of using such primers.

### 3. TECHNICAL BACKGROUND

Molecules of deoxyribonucleic acid (DNA) carry the genetic information for virtually all forms of life on earth. DNA is a polymer made up of smaller molecules called “nucleotides,” which contain a purine or pyrimidine base, a sugar moiety, and a phosphate group. The base portion of a nucleotide is a flat molecule containing carbon, nitrogen, hydrogen and, in most cases, oxygen. Pyrimidine bases are ring-shaped and may be cytosine (C) or thymine (T) Purine bases have a double ring shape and may be adenine (A) or guanine (G). The bases project from a sugar-phosphate backbone.

Under physiological conditions, DNA molecules are almost always double-stranded. The two strands of a DNA molecule are held together by chemical bonds that form between the bases on the two strands. Adenine pairs with thymine, and guanine pairs with cytosine. Thus, the two strands are not identical copies of each other; rather, the DNA strands of a double-stranded molecule are said to be “complementary.”

A DNA molecule may be separated into two single DNA strands by heat or by extremes of pH. The conditions under which a DNA molecule will separate (called “denaturation” or “melting”) depends in part on the length of the strands. For example, shorter strands typically denature at lower temperatures than longer strands. Upon return to physiological conditions, complementary strands will quickly rejoin. This process is called “renaturation.”

The unique sequence of bases along a DNA strand determines the genetic information of that strand. The ability to determine the sequence of bases on a DNA molecule has therefore been a tremendous boon to biology and medicine, and has also had significant consequences in other fields, such as criminology. In 1977, Allan Maxam and Walter Gilbert invented a chemical method for determining the base sequence of a DNA molecule. The Maxam and Gilbert method uses chemicals that specifically degrade DNA molecules in ways that allow researchers to determine the sequence.

Shortly after Maxam and Gilbert announced their method, Frederick Sanger introduced an enzymatic method to sequence DNA. In Sanger's method, one strand of a DNA molecule is annealed to a primer that is complementary to a portion of that strand. An enzyme, DNA polymerase, is added, which begins to produce a complementary copy of the DNA strand being sequenced. Molecules called “chain terminators” are added to the reaction mix to specifically inhibit the synthesis of the complementary copy in ways that allow researchers to determine the desired sequence.

DNA sequencing is now a routine and widely practiced technique. For example, efforts are currently underway to determine an entire human DNA sequence, which contains more than three billion bases. DNA sequencing also plays an important role in understanding genetic disease. By determining the DNA sequences of specific genes, scientists have identified numerous mutations (that is, alterations of the base sequence within a gene) that are associated with human diseases such as cancer, cystic fibrosis, and hemophilia. Researchers can use this information to develop diagnostic tests and, in some cases, therapies to treat persons at risk for genetic disease.

Traditional methods of DNA sequencing, however, suffer from a number of drawbacks. Significantly, the original methods for sequencing DNA do not work well when the amount of DNA to be sequenced is limited. Cycle sequencing, by comparison, allows the production of large amounts of product from relatively little template. In this technique, the DNA to be sequenced serves as the template for multiple rounds of primer extension. Cycling allows the production of more product than would be produced with the same quantity of template in a conventional sequencing reaction. Although the technique superficially resembles polymerase chain reaction (PCR), the goal is linear rather than exponential growth of the product.

This cycling regime, which is aimed at linear growth of the desired products, can also produce artifacts by exponential amplification of minor side-products. Any DNA strand produced that contains both a primer sequence and the complement of a primer sequence in the appropriate orientation will be a template for exponential amplification. Even if such molecules are produced only as rare side-products, their exponential growth may bring them to high concentrations relative to the linearly growing desired product. The problem potentially worsens as the number of cycles is increased. Other factors, such as the simultaneous use of two primers, may aggravate the problem. These artifacts can interfere with sequence determination.

Many of skill in the art have encountered problems with artifacts. Artifacts may appear when two primers are used to sequence outward from a transposable element. Resulting sequence ladders are heavily obscured by products that result from exponential amplification. Two unusual features of the primer system, namely the simultaneous presence of two primers and the partially pallindromic sequences of the transposon ends, contribute to the problem. However, artifacts are also observed when only one primer is used, and some templates yield artifacts in reactions not involving transposable elements. Attempts to combat these artifacts by adjustment of temperature profiles and ionic strength have not lead to consistently artifact-free sequence.

From the foregoing, it will be appreciated that it would be an advancement in the art to provide primers for use in cycle sequencing that do not produce exponential amplification artifacts. It would be a further advancement in the art to provide methods whereby linear amplification of a DNA sequence could occur without encountering exponential amplification artifacts.

Such primers and methods are disclosed herein.

### 4. BRIEF SUMMARY OF THE INVENTION

The present invention relates to primers for linear amplification of a nucleic acid template using a nucleic acid polymerase without the production of undesired artifacts. This is achieved by using a primer that cannot be replicated by the nucleic acid polymerase. In certain embodiments of the invention, the primer comprises an oligo(ribonucleotide) at the 5′ end and an oligo(deoxyribonucleotide) having one or more deoxyribonucleotide residues at the 3′ end. The oligo(ribonucleotide) may comprise RNA residues or hydrolysis-resistant oligo(ribonuclotide) residues such as oligo(2′-O-methylriboncleotide) residues. The primer may have at least about 2, 4, 5, or 6 deoxyribonucleotide residues. The invention may also be practiced with other primers incapable of being replicated by a DNA polymerase, such as a primer with at least 1 abasic residue.

The present invention also relates to methods of performing a linear amplification reaction without the production of undesired artifacts using a primer that cannot be replicated by a DNA polymerase. These methods include those related to use of the primers discussed above.

These and other advantages of the present invention will become apparent upon reading the following detailed description and appended claims.

### 5. BRIEF DESCRIPTION OF THE DRAWINGS

A more particular description of the invention briefly described above will be rendered by reference to the appended drawings and graphs. These drawings and graphs only provide information concerning typical embodiments of the invention and are not therefore to be considered limiting of its scope.

FIG. 1 schematically depicts a model of the failure of polymerase chain reaction using primers containing 2′-O-Me RNA residues.

FIG. 2 depicts PCR reactions of RNA-DNA chimeric primers. Three UP, RP primers with pGEM-3Xf(+) (Promega) and six S26, K26 series primers with KS52 as the template were used in a PCR reaction and then analyzed for products. Lane 1, UP-, RP-DNA primers; lane 2, UP-, RP-10d primers; lane 3, UP-, RP-5d primers; lane 4, S26-, K26-DNA primers; lane 5, S26d6-, K26d6-1 primers; lane 6, S26d6-, K26d6-2; lane 7, S26k6-, K26d6-3; lane 8, S26-, K26-DNA primers; land 9, S26-, K26-10d primers; lane 10, S26-K26-6d. L=1 μg BRL 123 bp ladder.

FIG. 3A is an autoradiograph showing the sequence of a plasmid template generated using the S26 series of primers. The sequence of the primers used are listed in Table 1. FIG. 3B is an autoradiograph showing the sequence of a plasmid template generated using the UP and RP series of primers.

FIG. 4 illustrates long and short primer extension times. This autoradiograph shows sequencing assays using the four S26 series chimeric primers. The L lanes refer to a cycling profile of 15 sec 94° C.; 4 min 60° C.; 4 min 72° C. The S lanes refer to a cycling profile of 15 sec 94° C.; 15 sec 60° C.; 1 min 72° C.

FIG. 5 is an autoradiograph showing the results of a two primer sequence reaction. In each reaction with S26, K26 series, the K26 type or primer was unlabeled. In each reaction with UP, RP series, the RP type of primer was unlabeled.

FIG. 6A schematically depicts a two-step extension experiment. Primers are represented by a bar with both DNA sections (open) and 2′-O-Me RNA sections (shaded). Location of the [γ-³²P] label is indicated by the asterisk.

FIG. 6B is a autoradiograph showing the results of a K26-DNA primer extended into a template containing 2′-O-Me RNA. The template was made from S26-DNA primer (lane 1), S26-12d (lane 2), S26-10d (lane 3), S26-8d (lane 4), S26-6d (lane 5), S26-5d (lane 6), S26-4d (lane 7), S26-2d (lane 8). L=Sequencing ladder from UP-primed M13. The length of extension from the end of the primer is noted at right.

FIG. 7 is a graph showing the melting temperatures (Tₘ, °C.) of DNA and modified primers.

FIG. 8 is a autoradiograph showing the results of sequence reactions using primers containing abasic residues. Lanes 1-3 are two-primer reactions with the primer pairs designated above the lanes. Lanes 4-6 are sequencing ladders from only one primer. The DNA lane is a control with two DNA primers. The 6d lane contains the S26-6d and K26-6d primer pair.

### 6. DETAILED DESCRIPTION OF THE INVENTION

The present invention provides primers for linear amplification of a nucleic acid template that are not subject to exponential amplification of undesired artifacts. The term “primer” is used herein in its ordinary sense to include single-stranded oligonucleotides that are used to “prime” or initiate polymerization by a nucleic acid polymerase of a complementary copy of a nucleic acid template. The term “primer” also includes other molecules, such as peptide nucleic acids, that can “prime” or initiate such polymerization. From the discussion below, it will be appreciated that a primer may comprise deoxyribonucleotides, ribonucleotides, and/or modified deoxyribo- and ribonucleotides. A primer of the present invention may be of any length, depending on the particular application, but is preferably between about 10 and about 100 nucleotides in length, more preferably between about 15 and about 50 nucleotides in length, and most preferably between about 17 and about 30 nucleotides in length.

In certain embodiments, the present invention provides a primer for linear amplification of a nucleic acid template using a nucleic acid polymerase, wherein the primer comprises a plurality of residues and wherein at least one of the residues in not a classical deoxyribonucleotide, such that the primer can not serve as an efficient template for polymerization. The term “classical deoxyribonucleotide” refers to 2′-deoxyadenosine (dA), 2′-deoxycytidine (dC), 2′-deoxyguanosine (dG), and 2′-deoxythymidine (or, more simply, “thymidine,” since this term specifically refers to a 2′-deoxyribonucleotide; dT). Thus, the phrase “not a classical deoxyribonucleotide” includes, for example, deoxyribonculeotides having modified or nonstandard bases, abasic residues, ribonucleotides, and modified ribonucleotides (such as 2′-O-methyl-ribonucleotides, 2′-fluoro-ribonucleotides, and 2′-amino-ribonucleotides). The phrase “can not serve as an efficient template for polymerization by the nucleic acid polymerase” means that the primer, as a template for replication, exhibits a decreased efficiency of polymerization by the nucleic acid polymerase when compared to the efficiency of polymerization of a comparable template composed entirely of deoxyribonucleotides. Preferably, the efficiency of polymerization is decreased by at least about 50%, more preferably by at least about 90%, and most preferably by about 99%.

In certain embodiments off the present invention, the nucleic acid template is DNA. The term “nucleic acid polymerase” as used herein encompasses both DNA and RNA polymerases. In certain embodiments of the invention, the nucleic acid polymerase is a DNA polymerase. The term “DNA polymerase” includes both DNA-dependent and RNA-dependent DNA polymerases. In certain preferred embodiments, the polymerase is a thermostable DNA polymerase. In certain especially preferred embodiments, the polymerase is Taq DNA polymerase.

In certain embodiments, a primer of the present invention may include at least one ribonucleotide residue. In certain other embodiments, a primer may include at least one 2′-O-methyl-ribonucleotide. In yet other embodiments, a primer of the present invention may include at least one abasic residue. The present invention also provides RNA primers for linear amplification.

The present invention also provides primers for linear amplification of a nucleic acid template using a DNA polymerase wherein the primer can not be replicated by the DNA polymerase. In certain embodiments of the present invention, the primer comprises a plurality of ribonucleotide residues at its 5′ end and at least two deoxyribonucleotide residues at its 3′ end. In certain embodiments, the primer may include at least about four, five, or six nucleotide residues at its 3′ end. In certain preferred embodiments, the ribonucleotide residues are 2′-O-methyl-ribonucleotides. In certain other embodiments, the primer includes at least one abasic residue.

The present invention also provides primers that comprise a block copolymer comprising an oligo(ribonucleotide) at the primer's 5′ end and an oligo(deoxyribonucleotide) at the primer's 3′ end. In certain embodiments, the primer comprises at least about two, four, five, or six deoxyribonucleotide residues. In certain other embodiments, the oligo(ribonucleotide) is hydrolysis-resistant. In certain preferred embodiments, the oligo(ribonucleotide) is an oligo(2′-O-methyl-ribonucleotide).

The present invention also provides methods for linear amplification of a nucleic acid template using a nucleic acid polymerase. Such methods include an annealing step, in which a primer that can not serve as an efficient template for polymerization by the nucleic acid polymerase is annealed to the template to form a template/primer duplex, and an incubation or extension step, in which the template/primer duplex is incubated with the nucleic acid polymerase. Such methods may also include a denaturation step in which the duplex is denatured or “melted.” The template may then be once again annealed to a primer for a further round of amplification.

In certain preferred embodiments, the nucleic acid template is DNA. The polymerase is preferably a DNA polymerase, more preferably a thermostable DNA polymerase, and most preferably Taq DNA polymerase.

Primers used in the methods of the present invention may be as described above. That is, such primers my comprise a block copolymer comprising an oligo(ribonucleotide) at the primer's 5′ end and an oligo(deoxyribonucleotide) at the primer's 3′ end. In certain preferred embodiments, the oligo(ribonucleotide) is hydrolysis-resistant. In certain especially preferred embodiments, the oligo(ribonucleotide) is an oligo(2′-O-methyl-ribonucleotide). In certain other embodiments, the primer may be RNA. In yet other embodiments, the primer may contain at least one abasic residue.

Exponential amplification, unlike linear amplification, relies on the fact that the product of one round of replication is a template for subsequent rounds. Part of this requirement is that the primer incorporated into a newly synthesized strand can later be replicated to form a primer binding site. No such requirement exists for linear growth. The use of a primer that cannot be replicated by DNA polymerase allows the desired linear amplification but prevents the undesired exponential growth of side-products.

Most DNA replication in vivo is initially primed by RNA, and RNA is not a template for a strictly DNA-dependent DNA polymerase. RNA therefore meets the criteria of a molecule that can serve as primer but not as template. However, using RNA as a primer presents a potential problem since RNA is susceptible to hydrolysis, both spontaneous and enzyme-catalyzed. Hydrolysis-resistant derivatives of RNA have been developed. One such derivative is 2′-O-methyl RNA (2′-O-Me RNA). In one embodiment of the invention, oligonucleotides comprising 2′-O-Me RNA residues are used as primers for linear amplification, including cycle sequencing. If a sufficient number of DNA residues are placed at the 3′ end of the oligonucleotide, linear amplification is efficient, but artifacts due to exponential amplification disappear. Another type of non-replicatable primer, a DNA oligomer containing one or more abasic residues, can also be used. The present invention also provides other types of non-replicatable primers such as primers in which one or more residues have modifications to the backbone sugar, such as 2′-F-RNA, 2′-amino-RNA, and arabinoside residues; primers comprising modifications to the backbone phosphate, such as methyl-phosphonate and H-phosphonate linkages; and entirely different backbone structures, such as that found in peptide nucleic acids (PNAs). The present invention also provides primers that have modified base structures, such as abasic residues or a 4-methylindole base.

Oligonucleotides containing 2′-O-Me RNA residues or abasic residues can serve as efficient primers for DNA synthesis by Taq DNA polymerase in cycle sequencing reactions. Moreover, the use of these primers prevents the appearance of the exponential amplification artifacts that are important to eliminate in cycle sequencing.

When using Taq polymerase, the ability of the 2′-O-methyl RNA oligonucleotides to prime DNA synthesis is dependent on the inclusion of a few DNA residues at the 3′ end of the molecule. With six or more DNA residues, priming efficiency was indistinguishable from that observed with conventional DNA primers. As the number of DNA residues is lowered below six in some cases, or five in others, the efficiency of primer extension falls off. This result can be reconciled with the crystal structure of Taq polymerase complexed with duplexDNA, in which the protein contacts the primer strand on only the last five residues of its 3′ end.

RNA (without DNA residues) is known to be an efficient primer for many DNA polymerases, including E. coli DNA polymerase I, a homolog of Taq polymerase. Taq polymerase appears to use RNA less efficiently as a primer, though a single 3′-terminal RNA residue does not interfere with PCR. 2′-O-Me RNA is even more different chemically from DNA, and probably presents more of a problem for primer extension. The need for DNA residues at the 3′ end of the primer is increased by methylation of the 2′-hydroxyl.

It is known that Taq polymerase possesses some reverse transcriptase (RTase) activity. This activity might compromise the beneficial effects of RNA primers. However, the RTase activity of Taq is weak, unlike that of the related Tth polymerase, and both enzymes show significant RTase activity only in the presence of manganese. If RTase activity were a potential problem, the use of 2′-O-Me RNA would have solved it. If a 2′-hydroxyl group makes for a bad replication template, methylation of this hydroxyl group should decrease replication efficiency further. It is expected, however, that the use unmodified RNA would also prevent artifact amplification.

Oligonucleotides containing DNA and 2′-O-Me RNA residues were found to form more stable duplexes with DNA as the number of 2′-O-Me RNA residues increased. DNA/2′-O-Me RNA hybrids can be more or less stable than the corresponding DNA/DNA hybrids, depending on sequence. In contrast, a stabilizing effect of 2′-O-Me RNA using four series of oligonucleotides with unrelated sequences was observed. The stabilizing effect may occur because of differences in the hybridization conditions. The largest difference in melting temperature between an oligonucleotide containing 2′-O-Me RNA and the corresponding all-DNA oligonucleotide was 7° C. This difference is smaller than the variation of melting temperatures among DNA primers of different sequences. The difference in melting behavior could be accommodated by a change of annealing temperatures or eliminated by adjustment of ionic strength or magnesium ion concentration. The cycle sequencing protocol has not been altered in order to use the modified primers.

Some modified primers of another type, DNA oligomers containing abasic sites near the 3′ end, are also capable of priming DNA synthesis efficiently. The primers containing one to three abasic sites located nine bases from the 3′ end all primed synthesis as effectively as a molecule with no abasic sites. However, when the abasic sites are located closer to the 3′ end, priming efficiency is lowered in some cases. Molecules containing two or three abasic sites located six residues from the 3′ end yields very little sequencing product. Although these sites would not directly contact the Taq polymerase (4), the modification must alter the primer-template structure enough to lower polymerization efficiency. In contrast, a single abasic site located six residues from the 3′ end does not lower priming efficiency. This modification does, however, prevent exponential amplification and therefore eliminated the artifact.

There is much latitude in the choice of primer types from the families of primers investigated. With DNA/2′-O-Me RNA oligonucleotides, as few as five or six DNA residues are sufficient for highly efficient priming, yet inclusion of as many as ten DNA residues does not lead to artifact amplification. An entirely different type of modification, the inclusion of abasic sites, may also be used. Enzymes other than Taq polymerase can likely be accomodated, and other modified oligonucleotides, or molecules with non-nucleotide linkages such as peptide nucleic acids, may play the same role as oligonucleotides containing 2′-O-Me RNA or abasic sites.

The use of non-replicatable primers appears to be a practical method of eliminating cycle sequencing artifacts. This method is both cost-competitive and convenient in that it requires no change in sequencing protocols other than the substitution of one primer for another. Although artifacts are most problematic when two primers are used together, they also arise when just one primer is used. The effect of using two primers may be template-specific; the addition of a second primer increases the combinatorial possibilities for exponential amplification by only a factor of four. Furthermore, two-primer systems are in use, and developments in fluorescent dye technology may lead to increased use of such systems. Modified primers should be particularly useful in applications where it is desirable to perform a large number of cycles, such as direct sequencing of microbial genomic DNA and direct sequencing of bacterial artificial chromosomes (BACs). The latter is essential to strategies involving BAC end-sequencing, which have become important in the human genome project. By allowing increases in number of cycles without accompanying artifact generation, modified primers may allow further advances in sequencing and related methodologies.

Table 1 lists the series of oligonucleotides used as primers in certain embodiments of the present invention. Lowercase letters within a primer sequence indicate positions with 2′-O-Me-phosphoramidites as discussed in the text. Dashes (−) within a primer sequence indicate abasic DNA sites. These oligonucleotides may be synthesized from commercially available dA, dG, dC, dT phosphoramidites (Perkin Elmer), 2′-O-Me-A, 2′-O-Me-G, 2′-O-Me-C, 2′-O-Me-U phosphoramidites (Glen Research), and dSpacer phosphoramidites (Glen Research) with an Applied Biosystems 394 DNA/RNA Synthesizer (Perkin Elmer). Methods of synthesizing oligonucleotides are known to those of skill in the art.

TABLE 1
Primer Sequence

| | |
|---|---|
| S26-DNA | |
| ATGACGCGCCGCTGTAAAGTGTTAGT | (SEQ ID NO:1) |
| S26-12d | |
| augacgcgccgcugTAAAGTGTTAGT | (SEQ ID NO:2) |
| S26-10d | |
| augacgcgccgcuguaAAGTGTTAGT | (SEQ ID NO:3) |
| S26-8d | |
| augacgcgccgcuguaaaGTGTTAGT | (SEQ ID NO:4) |
| S26-6d | |
| augacgcgccgcuguaaaguGTTAGT | (SEQ ID NO:5) |
| S26-5d | |
| augacgcgccgcuguaaagugTTAGT | (SEQ ID NO:6) |
| S26-4d | |
| augacgcgccgcuguaaaguguTAGT | (SEQ ID NO:7) |
| S26-2d | |
| augacgcgccgcuguaaaguguuaGT | (SEQ ID NO:8) |
| S26d6-1 | |
| ATGACGCGCCGCTGTAAAG-GTTAGT | (SEQ ID NO:9) |
| S26d6-2 | |
| ATGACGCGCCGCTGTAAA--GTTAGT | (SEQ ID NO:10) |
| S26d6-3 | |
| ATGACGCGCCGCTGTAA---GTTAGT | (SEQ ID NO:11) |
| S26d9-1 | |
| ATGACGCGCCGCTGTA-AGTGTTAGT | (SEQ ID NO:12) |
| S26d9-2 | |
| ATGACGCGCCGCTGT--AGTGTTAGT | (SEQ ID NO:13) |
| S26d9-3 | |
| ATGACGCGCCGCTG---AGTGTTAGT | (SEQ ID NO:14) |
| K26-DNA | |
| CTATAACGGTCCTAAGGTAGCGAGGT | (SEQ ID NO:15) |
| K26-10d | |
| cuauaacgguccuaagGTAGCGAGGT | (SEQ ID NO:16) |
| K26-6d | |
| cuauaacgguccuaagguagCGAGGT | (SEQ ID NO:17) |
| K26d6-1 | |
| CTATAACGGTCCTAAGGTA-CGAGGT | (SEQ ID NO:18) |
| K26d6-2 | |
| CTATAACGGTCCTAAGGT--CGAGGT | (SEQ ID NO:19) |
| K26d6-3 | |
| CTATAACGGTCCTAAGG---CGAGGT | (SEQ ID NO:20) |
| UP-DNA | |
| TAACGCCAGGGTTTTCCCAGTCACGA | (SEQ ID NO:21) |
| UP-10d | |
| uaacgccaggguuuucCCAGTCACGA | (SEQ ID NO:22) |
| UP-5d | |
| uaacgccaggguuuucccaguCACGA | (SEQ ID NO:23) |
| RP-DNA | |
| GTGAGCGGATAACAATTTCACACAGG | (SEQ ID NO:24) |
| RP-10d | |
| gugagcggauaacaauTTCACACAGG | (SEQ ID NO:25) |
| RP-5d | |
| gugagcggauaacaauuucacACAGG | (SEQ ID NO:26) |

For studies of melting behavior, DNA oligomers complementary to the primers were synthesized: antiFK,

| | |
|---|---|
| ACCTCGCTACCTTAGGACCGTTATAG; | (SEQ ID NO:27) |
| antiFS, | |
| ACTAACACTTTACAGCGGCGCGTCAT; | (SEQ ID NO:28) |
| antiUP, | |
| TCGTGACTGGGAAAACCCTGGCGTTA; | (SEQ ID NO:29) |
| and antiRP | |
| CCTGTGTGAAATTGTTATCCGCTCAC. | (SEQ ID NO:30) |

The template for extension studies was KS52,

| | |
|---|---|
| CTATAACGGTCCTAAGGTAGCGAGGTACTAACA | (SEQ ID NO:31) |
| CTTTACAGCGGCGCGTCAT. | |

### 7. EXAMPLES

The following examples are given to illustrate various embodiments which have been made within the scope of the present invention. It is to be understood that the following examples are neither comprehensive nor exhaustive of the many types of embodiments which can be prepared in accordance with the present invention.

### Example 1

### Polymerase Chain Reaction (PCR) with Modified Oligonucleotides

Amplification consisted of 25 cycles of 15 seconds at 96° C., 15 seconds at 60° C., and 3 minutes at 72° C. in an MJ Thermocycler. Reactions were carried out in 20 μl containing 100 fmol of template, 10 pmol of each primer, 200 μM dNTPs, 1.4 μg of Taq DNA polymerase modified as described in S. Tabor & C. C. Richardson, Proc. Natl. Acad. Sci. USA 92:6339-6343 (1995), 0.01 units of pyrophosphatase from Thermoplasma acidophilum, 2.75 mM MgCl₂, 10 mM Tris-HCl, pH 9.2, and 100 mM KCl. The template for the UP-RP series of primers was pGEM-3Zf(+) and for the S26-K26 series was KS52. Reactions were analyzed on 3% Nusieve GTG gels (FMC).

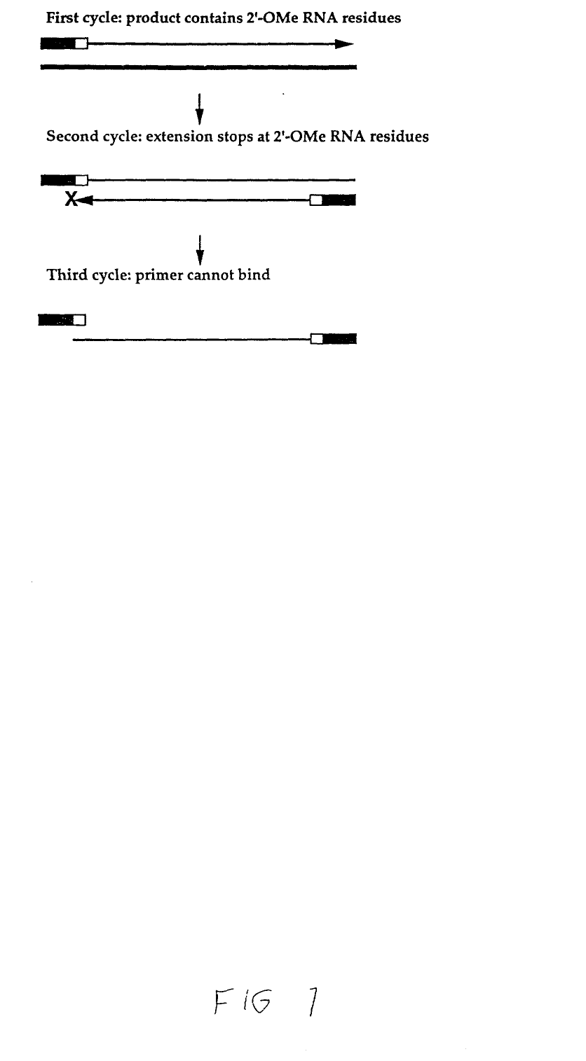

Referring to FIG. 1, a model of the failure of PCR using primers containg 2′-O-Me RNA residues is presented. The solid dark lines represent the template. The boxed areas are the primers. The open boxed areas are DNA residues of the primer. The shaded boxed areas are 2′-O-Me RNA residues of the primer.

The inclusion of 2′-O-Me RNA residues or abasic sites in primers prevents exponential amplification with Taq DNA polymerase. To verify this, several PCRs with conventional and modified primers were attempted. Modified Taq DNA polymerase, referred to herein as “Taq DNA polymerase,” was used throughout this study. The primer pairs used were forms of either UP and RP as specified in Table 1, the common “universal” and “reverse” primers found in many vectors, or S26 and K26, both specific for primer-binding sites on a transposon. The primer sets were standard DNA oligomers, chimeric oligonucleotides with both DNA and 2′-O-Me RNA residues, or DNA oligomers containing one or more abasic sites. Each oligonucleotide contained a total of 26 residues. The first type of modified primers contained 2 to 12 DNA residues at the 3′ end, the remaining residues being 2′-O-Me RNA. These oligonucleotides were named according to the number of DNA residues at the 3′ end. S26-4d, for example, contains 4 DNA residues at the 3′ end, and the other 22 residues are 2′-O-Me RNA. The second type of modified primers contained a stretch of one, two, or three abasic sites located six residues from the 3′ end. These oligonucleotides were named according to the number and position of abasic sites. S26d6-3, for example, contains six unmodified (ordinary DNA) residues at the 3′ end, followed, in the 3′ to 5′ direction, by three abasic residues and then seventeen unmodified residues to complete the primer.

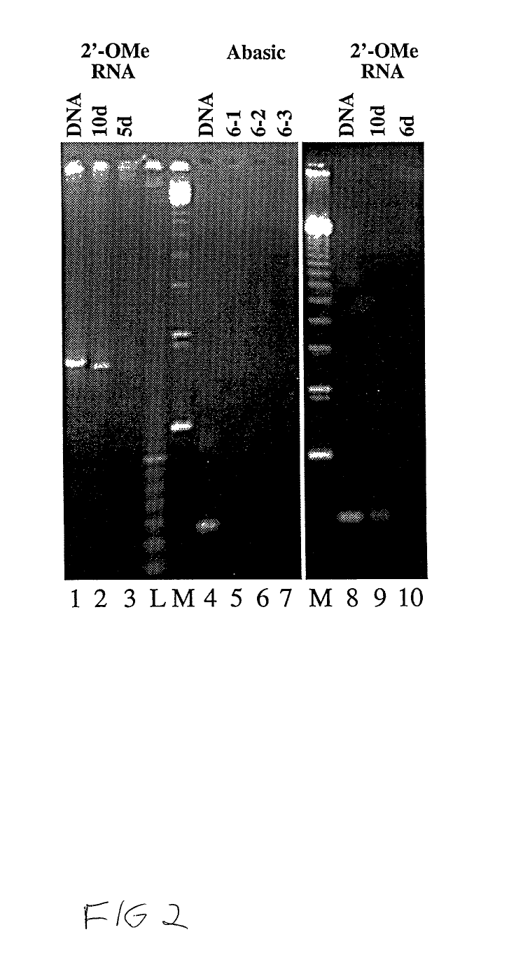

FIG. 2 compares the results of PCR using nine pairs of oligonucleotides: two pairs of ordinary DNA primers and seven pairs of modified primers. The first set of primers, UP and RP, was tested with the template pGEM-3Zf(+). As expected, a PCR product of 212 bp was observed when the primers contained all DNA (lane1). The next two lanes show the results when primers containing mostly 2′-O-Me RNA residues are utilized. When the UP- and RP-10d primers are used (lane 2) the amount of product is reduced. The UP-/RP-5d primer set does not yield any detectable product (lane 3)

PCR reactions using primers with abasic sites are shown in the next set of experimental lanes of FIG. 2. The template for the S26 and K26 primers in this experiment was a single-stranded oligonucleotide, KS52. The sequence of this 52-mer corresponds to the K26 primer followed by the complement of the S26 primer. It should therefore serve as a template for PCR with S26 and K26 primers. When S26- and K26-DNA primers are used a band is present at the expected position (lane 4). The other primers contain one (lane 5), two (lane 6), or three (lane 7) abasic sites. There appears to be no product in any of the reactions using two primers with abasic sites.

The last set of lanes shows the results using S26 and K26 primers with 2′-O-Me RNA residues. The template for these PCRs was KS52. When S26- and K26-DNA primers are used, a band is present at the expected position (FIG. 2, lane 8). The other primers contain either 10 (lane 9) or 6 (lane 10) DNA residues at the 3′ end. There appears to be less product in lane 9, and very little product is visible in lane 10. These results demonstrate that the modified oligonucleotides do not support efficient exponential amplification.

### Example 2

### DNA Sequencing with Chimeric Primers

In order to determine the relative efficiencies with which the chimeric DNA/2′-O-Me RNA oligonucleotides could be used as primers by Taq polymerase, sequencing reactions were attempted. Primers were end-labeled with [γ-³²P] ATP and T4 polynucleotide kinase. Unless otherwise noted, the thermal cycle sequencing reactions were performed as follows. Sequencing reaction mixes contained 25 fmol of template, 1.25 pmol of labeled primer, 2.75 mM MgCl₂, 10 mM Tris-HCl, pH 9.2, 100 mM KCl, 0.01 U of pyrophosphatase, and 1.4 μg of Taq polymerase, 125 μM of each dNTP and either ddATP, ddGTP, ddCTP, or ddTTP at 1 μM in a total volume of 20 μl. Thermal cycling consisted of 25 cycles of 10 seconds at 96° C., a 1°/second ramp to 50° C., 15 seconds 50° C., a 1°/second ramp to 60° C., and 4 minutes at 60° C. The template, pWD42a, is a 5.2 kb plasmid with an 8 kb insert and an RI origin of replication. Priming sites for the UP and RP primers flank the insert. They are oriented such that the primers extend toward each other and into the insert. This plasmid carries a γδ transposon that provides priming sites for the K26 and S26 primers. These priming sites are located 368 bp apart and are oriented in opposite directions such that primer extension from the two sites is divergent. Products were separated on 6% acrylamide gels.

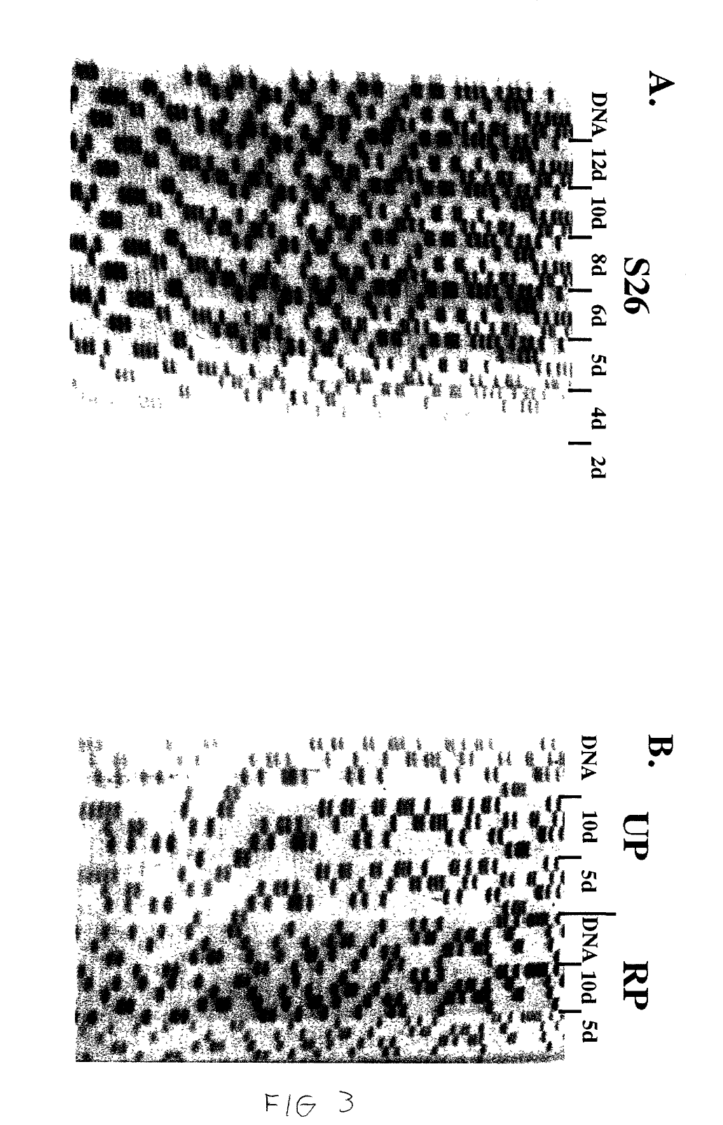

Each primer in the S26 2′-O-Me series was labeled with ³²P and used in a cycle sequencing reaction. As seen in FIG. 3A, the 12d, 10d, 8d, 6d, and 5d primers work as well as a conventional DNA primer. The 4d oligonucleotide is slightly less effective as a primer, and the 2d primer yields no detectable signal under these conditions. FIG. 3b shows a similar test of chimeric oligonucleotides with the UP and RP primer series. The DNA and chimeric versions of the UP primer appeared to be equally effective as sequencing primers. The DNA and 10d versions of the RP primer yielded equal product intensities, but the 5d sequence ladders were slightly less intense.

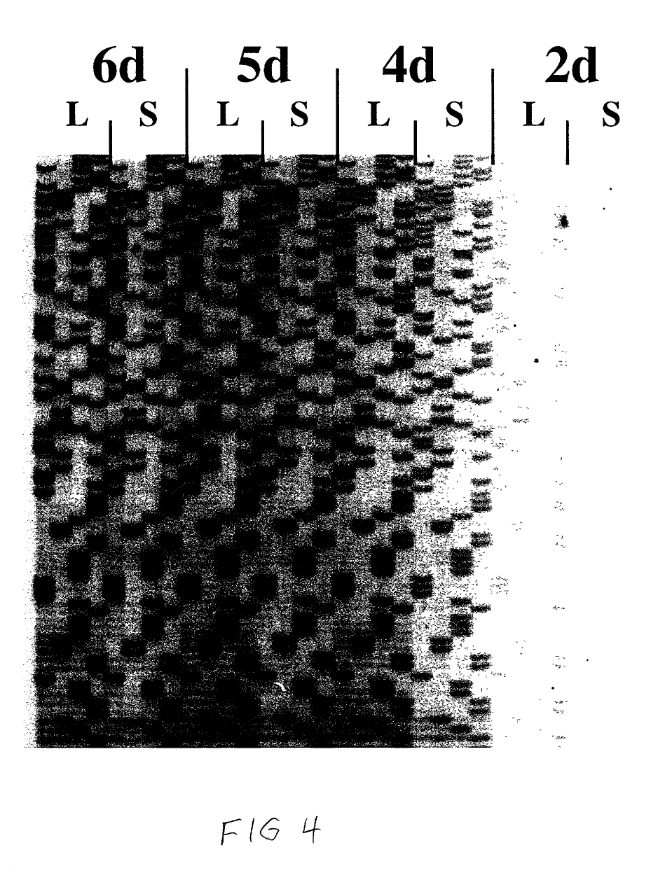

The primers with fewer than five DNA residues yielded light (S26-4d) or undetectable (S26-2d) sequencing ladders. This was presumably the result of inefficient extension of an unnatural substrate by Taq polymerase. If this were the case, increasing the annealing and extension times might increase the product yield. FIG. 4 shows a comparison using the S26-6d, 5d, 4d and 2d primers with two cycling protocols. The long (L) cycling conditions increased the annealing time from 15 seconds to 4 minutes and the extension time from 1 minute to 3 minutes as compared to the short (S) cycling conditions. The longer hybridization and extension times had no effect on the S26-6d and 5d primers, which perform as well as DNA primers even when cycle times are short. However, FIG. 4 shows that the 4d primer yields more product under the longer times. More striking is the case of the 2d primer, which yields no visible product under the short times but a clearly visible, albeit light, ladder under the longer hybridization and extension times.

### Example 3

### Artifact Elimination with Chimeric Primers

The above data suggest that the use of chimeric DNA/2′-O-Me RNA primers in a sequencing reaction might yield good sequencing ladders while preventing artifacts that are due to exponential amplification. This possibility was tested using several primer pairs on a single template. Two primers are used in each experiment. Each primer can hybridize to the template, but only one primer is labeled. Two informative sequencing ladders may be produced, but only one is visualized.

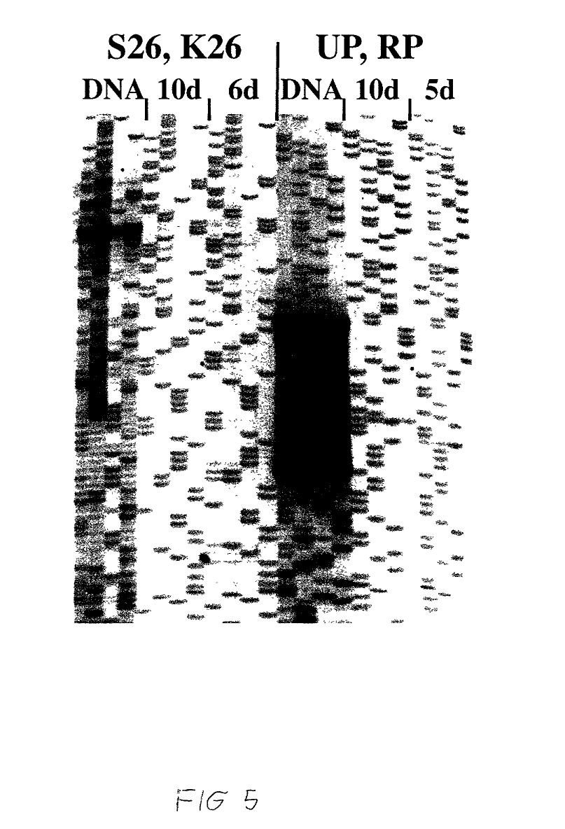

The left side of FIG. 5 shows the results from using the K26 and S26 primers together in a sequencing reaction. The template for the reaction was one that had led to a variety of artifact bands using the conventional DNA primers. In this experiment the ³²P-labeled S26-DNA primer and unlabeled K26-DNA primer were combined with pWD42a in a sequencing reaction. The sequencing ladder can be partially read but is greatly obscured by a number of undesired products. However, when ³²P-labeled S26-10d or S26-6d primers were used with unlabeled K26-10d or K26-6d respectively, the undesired products were eliminated and the entire sequence ladder could be clearly seen (S26/K26 10d and 6d ladders in FIG. 5).

An additional experiment tested the UP/RP primer series with the same template. The first lane in this series uses ³²P-labeled UP-DNA primer and unlabeled RP-DNA primer. This combination yields intense artifact bands, preceded by a double sequencing pattern. The result is a severely obscured sequencing ladder. The other two ladders on the right of FIG. 5 show similar experiments with the 10d and 5d primer pairs. In both cases it is the UP-type primer that is labeled with ³²P. Neither of these lane-sets shows any artifact but they do show good quality sequence ladders.

### Example 4

### Primer Extension Assays

Primer extension experiments were carried out in two stages. First, an extension reaction was carried out in a 20 μl reaction volume using 10 pmol of one of the S26 series primers, 50 fmol of KS52 template, 1.4 μg of Taq DNA polymerase with 2.75 mM MgCl₂, 10 mM Tris-HCl, pH 9.2, and 100 mM KCl in an MJ Thermocycler using 25 cycles of 96° C. for 15 seconds., 60° C. for 15 seconds., and 72° C. for 2 minutes. A second extension was then carried out after addition of 1.4 μg of Taq DNA polymerase, 5 pmol of labeled K26-DNA primer, and buffer to maintain ion concentrations and bring the total volume to 30 μl. Conditions for this extension reaction were the same as for the first, but only one cycle was performed.

As indicated above, some chimeric DNA/2′-O-Me RNA primers can both prevent exponential amplification and efficiently prime sequencing reactions. The ability of the chimeric primers to prevent exponential amplification is probably due to the inability of Taq polymerase to use 2′-O-Me RNA as a template. The polymerase might stop at the junction between DNA and 2′-O-Me RNA residues, or it might polymerize a few nucleotides into the 2′-O-Me region. In order to determine how the polymerase was behaving, primer extension assays were performed.

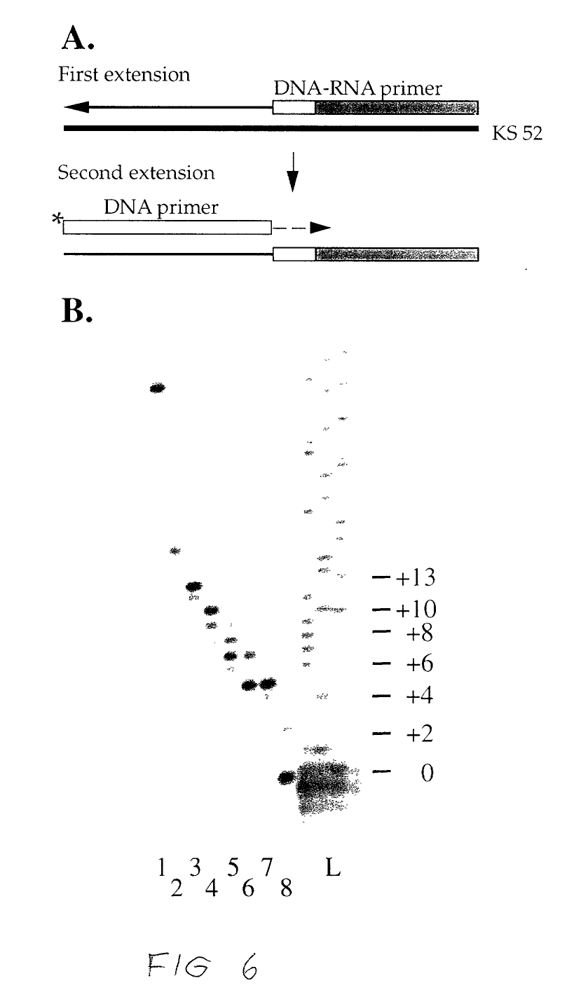

The primer extensions were performed in two stages. First, one of the S26 series primers was extended using KS52 as a template (FIG. 6a). The expected product would contain a priming site for the K26 primer. This would be followed, in the direction of K26 extension, by either all DNA residues or, if a chimeric primer was used in the first stage, several DNA residues and then 2′-O-Me RNA residues. This product then served as a template for extension of ³²P-labeled K26 (DNA) primer through the DNA region and potentially into the 2′-O-Me RNA residues.

The results of these experiments (FIG. 6b) show that although Taq polymerase can usually extend partway into the 2′-O-Me RNA region of a template, it stops within a few bases of the DNA/2′-O-Me RNA junction. Each DNA/2′-O-Me RNA template yielded a range of products in the second extension, but one predominant band. The full-length product from the all-DNA control is clearly seen (FIG. 6b, lane 1). With one exception (the experiment using 2d), when templates containing 2′-O-Me RNA were used the DNA primer was extended at least up to the DNA/2′-O-Me RNA junction, and usually beyond. When the template incorporated the 4d, 5d, 6d, 8d, 10d, or 12d oligonucleotide, the primer was extended 1, 0, 1, 2, 2, or 3 bases beyond the DNA/2′-O-Me RNA junction, respectively. Some of this variation must represent local sequence effects, although the amount of upstream DNA may also have some influence on extension. When the template contained only two DNA residues past the priming site, little primer extension was observed (FIG. 6b, lane 8). This result suggests that Taq polymerase cannot efficiently initiate synthesis two bases upstream of the 2′-O-Me RNA, despite the fact that it can apparently extend up to the 2′-O-Me RNA, or beyond, if it has initiated synthesis further upstream.

### Example 5

### Hybridization of DNA/2′-O-Me RNA Oligonucleotides to DNA

Modifications to the backbone of an oligonucleotide will affect its hybridization properties. In order to assess the effect of 2′-O-Me RNA residues, the melting temperatures (Tₘ) of duplexes consisting of a primer (DNA or chimeric) hybridized to a complementary DNA strand were determined. Melting curves were measured as follows. Absorption at 260 nm was monitored on a Beckman DU 7400 spectrophotometer over a range of temperatures. Equimolar amounts of sample and antisense oligonucleotide were placed in a buffer containing 2.75 mM MgCl₂, 10 mM Tris pH 9.3, and 100 mM KCl. The temperature profile was 35-65° C., 1°/min; 66-85° C., 0.5°/min; and 86-94° C., 1°/min. Melting temperature was taken to be the temperature at which the first derivative was highest.

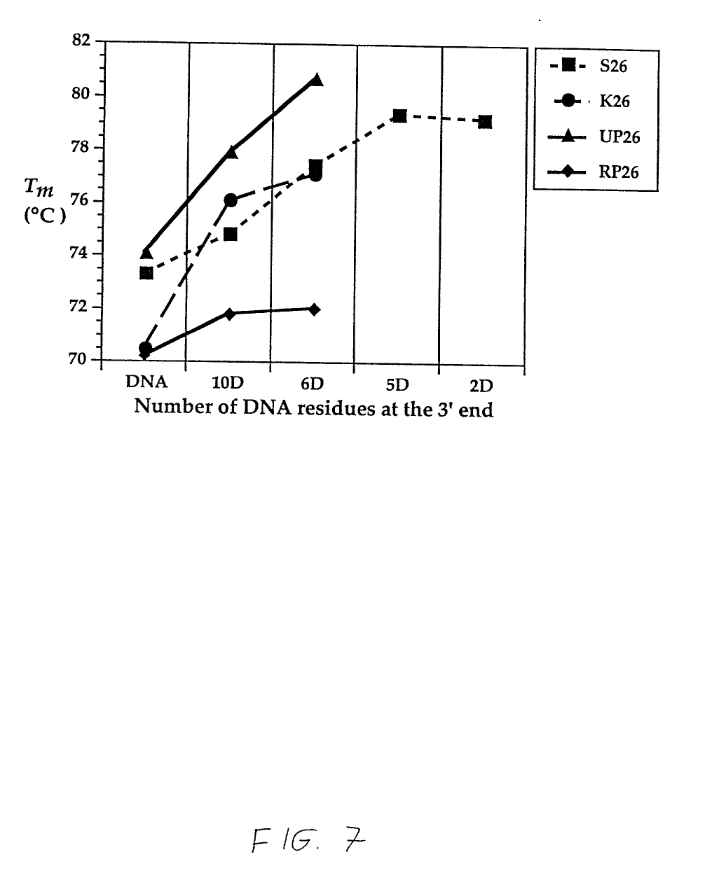

FIG. 7 shows the measured Tₘ for each chimeric oligonucleotide used in this study. The melting temperatures for both the UP and K26 series rise by about 7° C. when 20 of the DNA residues are replaced with 2′-O-Me RNA residues. The increase in Tₘ for the S26series is not quite as large, but it does increase and levels off at the point where 21 DNA residues have been replaced by 2′-O-Me RNA residues. For the RP series, the Tₘ increased by only about 2° C., several degrees less than for the other series. The Tₘ generally increases with the number of 2′-O-Me RNA residues present in the oligonucleotide.

### Example 6

### Sequencing with Primers Containing Abasic Sites

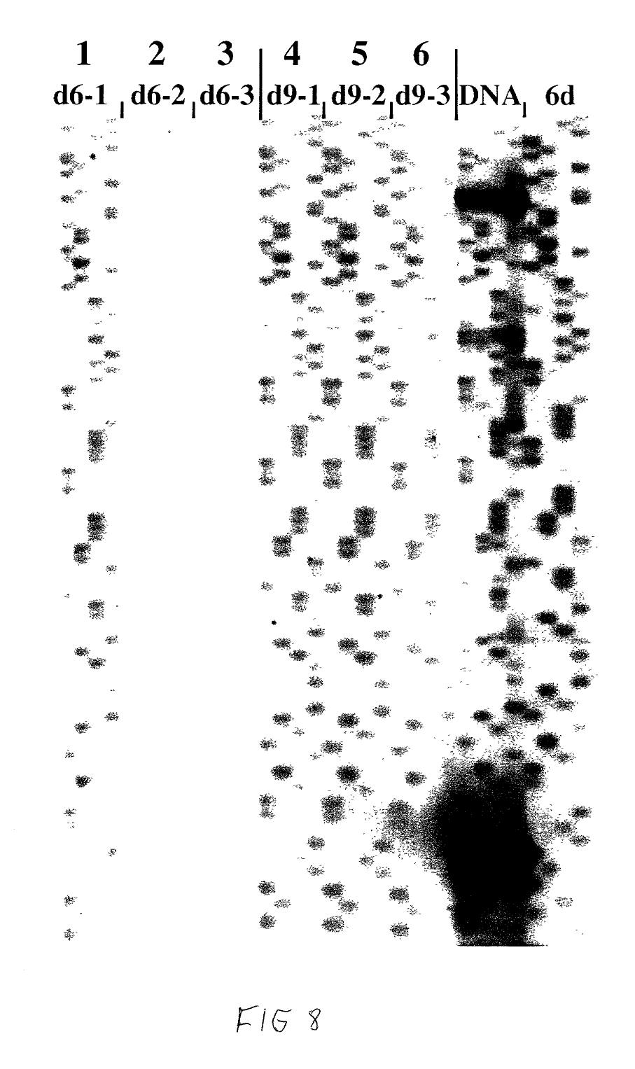

Abasic primers were tested for their ability to prime sequencing reactions and eliminate artifacts. The sequencing reaction was carried out according to the procedure set out for the 2′-O-Me RNA residues above. FIG. 8 illustrates the results of this experiment.

Lane 1 in FIG. 8 shows that oligomers containing a single abasic site located six bases from the 3′ end work effectively as sequencing primers. Furthermore, the artifact is eliminated by the use of these primers (compare lanes 1 and 7). Addition of more abasic sites results in a lower yield of sequencing products (lanes 2 and 3). When the abasic sites are placed further (nine residues) from the 3′ end, even oligonucleotides containing two (lane 4) or three (lane 5) abasic sites appear to work as well as unmodified DNA primers.

Summary

In summary, the present invention provides primers for use in linear amplification of a nucleic acid template by a nucleic acid polymerase that do not contribute to exponential amplification of artifacts. In particular, the present invention provides primers containing at least one residues that is not a deoxyribonucleotide. Such residues may be, for example, ribonucleotides, 2′-O-methyl ribonucleotides, or abasic residues. The present invention also discloses methods of linear amplification of a DNA template using such primers.

I claim:

1. A method of performing a linear amplification of a nucleic acid template using a nucleic acid polymerase comprising the steps of:
a. annealing a primer to the template to form a template/primer duplex, wherein the primer can not be serve as an efficient template for polymerization by the nucleic acid polymerase; and
b. incubating the template/primer duplex with the nucleic acid polymerase.

2. The method of claim 1, wherein the nucleic acid polymerase is a DNA polymerase.

3. The method of claim 2, wherein the DNA polymerase is a thermostable DNA polymerase.

4. The method of claim 3, wherein the thermostable DNA polymerase is Taq DNA polymerase.

5. The method of claim 2, wherein the primer comprises a 5′ end and a 3′ end, and wherein the primer further comprises a block copolymer comprising an oligo(ribonucleotide) at the 5′ end of the primer and an oligo(deoxyribonucleotide) at the 3′ end of the primer.

6. The method of claim 5, wherein the oligo(deoxyribonucleotide) comprises at least two deoxyribonucleotide residues.

7. The method of 6, wherein the oligo(ribonucleotide) is hydrolysis-resistant.

8. The method of claim 6, wherein the oligo(ribonucleotide) is an oligo(2′-O-methylriboncleotide).

9. The method of claim 5, wherein the oligo(deoxyribonucleotide) comprises at least four deoxyribonucleotide residues.

10. The method of claim 5, wherein the oligo(deoxyribonucleotide) comprises at least five deoxyribonucleotide residues.

11. The method of claim 5, wherein the oligo(deoxyribonucleotide) comprises at least six deoxyribonucleotide residues.

12. The method of claim 2, wherein the primer is RNA.

13. The method of claim 2, wherein the primer comprises at least 1 abasic residues.


SEQUENCE LISTING (31 sequences)

SEQ ID NO: 1 - DNA, 26 bp, Artificial Sequence
  Description of Artificial Sequence Synthetic Oligonucleotide
atgacgcgccgctgtaaagtgttagt

SEQ ID NO: 2 - DNA, 26 bp, Artificial Sequence
  Description of Combined DNA/RNA Molecule Residues 1-14 are 2′-O-methyl ribonucleotides; residues 15-26 are deoxyribonucleotides.
augacgcgccgcugtaaagtgttagt

SEQ ID NO: 3 - DNA, 26 bp, Artificial Sequence
  Description of Combined DNA/RNA Molecule Residues 1-16 are 2′-O-methyl ribonucleotides; residues 17-26 are deoxyribonucleotides.
augacgcgccgcuguaaagtgttagt

SEQ ID NO: 4 - DNA, 26 bp, Artificial Sequence
  Description of Combined DNA/RNA Molecule Residues 1-18 are 2′-O-methyl ribonucleotides; residues 19-26 are deoxyribonucleotides.
augacgcgccgcuguaaagtgttagt

SEQ ID NO: 5 - DNA, 26 bp, Artificial Sequence
  Description of Combined DNA/RNA Molecule Residues 1-20 are 2′-O-methyl ribonucleotides; residues 21-26 are deoxyribonucleotides.
augacgcgccgcuguaaagugttagt

SEQ ID NO: 6 - DNA, 26 bp, Artificial Sequence
  Description of Combined DNA/RNA Molecule Residues 1-21 are 2′-O-methyl ribonucleotides; residues 22-26 are deoxyribonucleotides.
augacgcgccgcuguaaagugttagt

SEQ ID NO: 7 - DNA, 26 bp, Artificial Sequence
  Description of Combined DNA/RNA Molecule Residues 1-22 are 2′-O-methyl ribonucleotides; residues 23-26 are deoxyribonucleotides.
augacgcgccgcuguaaagugutagt

SEQ ID NO: 8 - DNA, 26 bp, Artificial Sequence
  Description of Combined DNA/RNA Molecule Residues 1-24 are 2′-O-methyl ribonucleotides; residues 25-26 are deoxyribonucleotides.
augacgcgccgcuguaaaguguuagt

SEQ ID NO: 9 - DNA, 26 bp, Artificial Sequence
  Residue 20 is an abasic residue
atgacgcgccgctgtaaagngttagt

SEQ ID NO: 10 - DNA, 26 bp, Artificial Sequence
  Residues 19-20 are abasic residues
atgacgcgccgctgtaaanngttagt

SEQ ID NO: 11 - DNA, 26 bp, Artificial Sequence
  Residues 18-20 are abasic residues
atgacgcgccgctgtaannngttagt

SEQ ID NO: 12 - DNA, 26 bp, Artificial Sequence
  Residue 17 is an abasic residue
atgacgcgccgctgtanagtgttagt

SEQ ID NO: 13 - DNA, 26 bp, Artificial Sequence
  Residues 16-17 are abasic residues
atgacgcgccgctgtnnagtgttagt

SEQ ID NO: 14 - DNA, 26 bp, Artificial Sequence
  Residues 15-17 are abasic residues
atgacgcgccgctgnnnagtgttagt

SEQ ID NO: 15 - DNA, 26 bp, Artificial Sequence
  Description of Artificial Sequence Synthetic Oligonucleotide
ctataacggtcctaaggtagcgaggt

SEQ ID NO: 16 - DNA, 26 bp, Artificial Sequence
  Description of Combined DNA/RNA Molecule Residues 1-16 are 2′-O-methyl ribonucleotides; residues 17-26 are deoxyribonucleotides.
cuauaacgguccuaaggtagcgaggt

SEQ ID NO: 17 - DNA, 26 bp, Artificial Sequence
  Description of Combined DNA/RNA Molecule Residues 1-20 are 2′-O-methyl ribonucleotides; residues 21-26 are deoxyribonucleotides.
cuauaacgguccuaagguagcgaggt

SEQ ID NO: 18 - DNA, 26 bp, Artificial Sequence
  Residue 20 is an abasic residue
ctataacggtcctaaggtancgaggt

SEQ ID NO: 19 - DNA, 26 bp, Artificial Sequence
  Residues 19-20 are abasic residues
ctataacggtcctaaggtnncgaggt

SEQ ID NO: 20 - DNA, 26 bp, Artificial Sequence
  Residues 18-20 are abasic residues
ctataacggtcctaaggnnncgaggt

SEQ ID NO: 21 - DNA, 26 bp, Artificial Sequence
  Description of Artificial Sequence Synthetic Oligonucleotide
taacgccagggttttcccagtcacga

SEQ ID NO: 22 - DNA, 26 bp, Artificial Sequence
  Description of Combined DNA/RNA Molecule Residues 1-16 are 2′-O-methyl ribonucleotides; residues 17-26 are deoxyribonucleotides.
uaacgccaggguuuucccagtcacga

SEQ ID NO: 23 - DNA, 26 bp, Artificial Sequence
  Description of Combined DNA/RNA Molecule Residues 1-21 are 2′-O-methyl ribonucleotides; residues 22-26 are deoxyribonucleotides.
uaacgccaggguuuucccagucacga

SEQ ID NO: 24 - DNA, 26 bp, Artificial Sequence
  Description of Artificial Sequence Synthetic Oligonucleotide
gtgagcggataacaatttcacacagg

SEQ ID NO: 25 - DNA, 26 bp, Artificial Sequence
  Description of Combined DNA/RNA Molecule Residues 1-16 are 2′-O-methyl ribonucleotides; residues 17-26 are deoxyribonucleotides.
gugagcggauaacaauttcacacagg

SEQ ID NO: 26 - DNA, 26 bp, Artificial Sequence
  Description of Combined DNA/RNA Molecule Residues 1-21 are 2′-O-methyl ribonucleotides; residues 22-26 are deoxyribonucleotides.
gugagcggauaacaauuucacacagg

SEQ ID NO: 27 - DNA, 26 bp, Artificial Sequence
  Description of Artificial Sequence Synthetic Oligonucleotide
acctcgctaccttaggaccgttatag

SEQ ID NO: 28 - DNA, 26 bp, Artificial Sequence
  Description of Artificial Sequence Synthetic Oligonucleotide
actaacactttacagcggcgcgtcat

SEQ ID NO: 29 - DNA, 26 bp, Artificial Sequence
  Description of Artificial Sequence Synthetic Oligonucleotide
tcgtgactgggaaaaccctggcgtta

SEQ ID NO: 30 - DNA, 26 bp, Artificial Sequence
  Description of Artificial Sequence Synthetic Oligonucleotide
cctgtgtgaaattgttatccgctcac

SEQ ID NO: 31 - DNA, 52 bp, Artificial Sequence
  Description of Artificial Sequence Synthetic Oligonucleotide
ctataacggtcctaaggtagcgaggtactaacactttacagcggcgcgtcat